(19) United States
(12) Patent Application Publication
(10) Pub. No.: US 2014/0277485 A1
(43) Pub. Date: Sep. 18, 2014

(54) INTERVERTEBRAL FUSION IMPLANT CAGE

(71) Applicant: Ranier Limited, Cambridge (GB)

(72) Inventors: Scott Johnson, Newmarket (GB); Ian Hugh Quirk, Oxfordshire (GB)

(73) Assignee: Ranier Limited, Cambridge (GB)

(21) Appl. No.: 13/830,121

(22) Filed: Mar. 14, 2013

(51) Int. Cl. A61F 2/44

(52) U.S. Cl. CPC A61F 2/442
USPC 623/17.16

(57) ABSTRACT

Intervertebral fusion implant cages and related methods are described herein. The cages may be used to assist with fusing together adjacent vertebrae when a displaced or damaged spinal disc is surgically removed. The cages include windows that allow the bone graft to grow from one vertebra through the cage and into the adjacent vertebra (e.g., cervical vertebrae). The cages include a number of features which enhance its performance during use.

### FIELD OF INVENTION

The present invention relates generally to spinal implants and, more particularly, to intervertebral fusion implant cages and related methods.

### BACKGROUND OF INVENTION

A spinal disc lies between adjacent vertebrae in the spine. The disc stabilizes the spine and assists in distributing forces between vertebral bodies. A spinal disc includes an outer annulus fibrosis which surrounds an inner nucleus pulposus. The annulus fibrosis is a concentrically laminated structure of aligned collagen fibers and fibro cartilage which provides stability to resist axial torsional and bending forces. The nucleus pulposus comprises a gelatinous material which can distribute stresses acting on the disc.

A spinal disc may be damaged due to trauma, disease or other degenerative processes that can occur over time. For example, the annulus fibrosis may weaken and/or begin to tear which can result in the protrusion of the nucleus pulposus into a region of the spine (e.g., the vertebratal foramen) that includes spinal nerves. The protruding nucleus pulposus may press against spinal nerves causing pain, numbness, tingling, diminished strength and/or a loss of motion. Another common degenerative process is the loss of fluid from the nucleus pulposus. Such fluid loss can limit the ability of the nucleus pulposus to distribute stress and may reduce its height which can lead to further instability of the spine, as well as decreasing mobility and causing pain.

To address the conditions described above, a displaced or damaged spinal disc may be surgically removed from the spine and the two adjacent vertebrae may be fused together. Implant fusion cages may be used in such procedures to promote fusion. The cages are generally porous to allow the bone graft to grow from one vertebra through the cage and into the adjacent vertebra.

### SUMMARY OF INVENTION

Intervertebral fusion implant cages and related methods are described herein.

In one aspect, an intervertebral fusion implant cage designed to be implanted between adjacent vertebrae is provided. The implant cage comprises a curved superior surface shaped to match anatomical curvatures of an adjacent vertebra and a curved inferior surface shaped to match anatomical curvatures of an adjacent vertebra. The cage further comprises an anterior wall; a posterior wall; and, a first sidewall connecting the anterior wall to the posterior wall. The cage further comprises a second sidewall connecting the anterior wall to the posterior wall at a side opposite the first sidewall; and, an inner wall extending from the anterior wall to the posterior wall. The cage further comprises a first window extending from the superior surface to the inferior surface and defined between the inner wall, the first and second sidewalls, the anterior wall and the posterior wall; and, a second window extending from the superior surface to the inferior surface and defined between the inner wall, the first and second sidewalls, the anterior wall and the posterior wall.

Other aspects, embodiments and features of the invention will become apparent from the following detailed description of the invention, when considered in conjunction with the accompanying drawings. It should be understood that aspects of the invention are described herein with reference to the figures, which show illustrative embodiments of the invention. The embodiments described herein are not necessarily intended to show all aspects of the invention. It should be appreciated, then, that the various concepts and embodiments introduced above and those discussed in greater detail below may be implemented in any of numerous ways, as the disclosed concepts and embodiments are not limited to any particular manner of implementation. In addition, it should be understood that aspects of the invention may be used alone or in any suitable combination with other aspects of the invention. All patent applications and patents incorporated herein by reference are incorporated by reference in their entirety. In case of conflict, the present specification, including definitions (if any), will control.

### BRIEF DESCRIPTION OF THE DRAWINGS

The accompanying drawings are not intended to be drawn to scale. In the drawings, each identical or nearly identical component that is illustrated in various figures may be represented by a like numeral. For purposes of clarity, not every component may be labeled in every drawing. Various embodiments of the invention will now be described, by way of example, with reference to the accompanying drawings, in which:

FIGS. 1A-1E show respective views of an intervertebral fusion implant cage in accordance with embodiments.

### DETAILED DESCRIPTION

Intervertebral fusion implant cages and related methods are described herein. The cages may be used to assist with fusing together adjacent vertebrae when a displaced or damaged spinal disc is surgically removed. The cages include windows that allow the bone to grow from one vertebra through the cage and into the adjacent vertebra (e.g., cervical vertebrae). As described further below, the cages include a number of features which enhance its performance during use.

FIGS. 1A-1E show an intervertebral fusion implant cage 10 according to an embodiment. The implant cage includes a superior surface 12 at the top of the cage and an inferior surface 14 at the bottom of the cage. Such surfaces can be curved and, for example, shaped to match anatomical curvatures on an adjacent vertebra. The implant cage, as shown, includes several walls which define the periphery of the device. An anterior wall 15 (which is positioned on the anterior side of the cage once implanted) is connected to a posterior wall 16 (which is positioned on the posterior side of the cage once implanted) by sidewalls 18a, 18b. An inner wall 20 extends from the anterior wall to the posterior wall to define windows 22a, 22b. The windows extend from the superior surface to the inferior surface to allow bone to grow through the device. In some embodiments, the windows may be partially or completely filled with a bone graft and/or synthetic bone material for stimulating bone growth between the adjacent vertebra.

The fusion implant cage can be formed of polymeric material. In some cases, the polymeric material that forms the fusion implant cage is a polyurethane material (i.e., a polymeric material having a polyurethane component). Such polyurethane materials may also include other polymeric components such as polycarbonate (e.g., polyurethane polycarbonate materials). Linear, slightly or highly branched, cross-linked and interpenetrating networks polyurethane materials may be suitable. It should be understood that other compatible polymeric materials may be used to form the implant cage in some embodiments.

In some embodiments, substantially all of the fusion implant cage is formed of a polymeric material. In such embodiments, advantageously, the cage may be MRI compatible. For example, substantially all of the fusion implant cage may be formed of the same type of polymeric material. That is, a single type of material may be used to form substantially all of the fusion implant cage. In some cases, the fusion cage may be formed of the same type of polymeric material (e.g., a polyurethane material) with varying stoichiometry at different locations of the fusion cage. In some cases, the fusion cage may be formed of the same type of polymeric material (e.g., a polyurethane material) with different relative amounts of hard segments and soft segments at different locations of the fusion cage. For example, peripheral regions of the fusion cage may be formed of a polymeric material (e.g., a polyurethane material) having a first stoichiometry and/or a first relative amount of hard segments and soft segments), while interior regions may be formed of the same polymeric material composition having a second stoichiometry different than the first stoichiometry and/or a different relative amount of hard segments and soft segments. For example, the material in the peripheral regions may have a lower molecular weight than the material in the interior regions (or vice-versa) which results in the difference in stoichiometry. The difference in stoichiometry can also lead to differences in properties (e.g., compressive modulus) between the peripheral region and the interior region. For example, the interior region may be stiffer and/or have a higher compressive modulus than the peripheral region as a result of stoichiometry differences. In some embodiments, the stoichiometry of the polymeric material and/or relative amount of hard segments and soft segments may be graded across at least a portion of the cage which can also lead to the grade in properties.

In some embodiments, the fusion implant cage is formed of a foam material. In such embodiments, the implant cage may be formed of polymeric material as described above. The foam material includes a plurality of cells. In some embodiments, the foam may have an open cell structure which is characterized by the interconnection of some cells with one another. The degree of interconnection may vary depending on the embodiment. In some cases, the degree of interconnection is such that no fluid pathway is present which extends through the thickness of the open cell foam.

It should be understood that embodiments in which substantially all of the fusion implant cage is formed of a polymeric material, there may be small amounts (e.g., less than 5 weight % of the total weight of the cage, less than 3 weight % of the total weight of the cage, less than 1 weight % of the total weight of the cage; e.g., less than 2% volume of the device) of other types of materials such as materials used as radiographic markers, which are described further below.

In some embodiments, it may be preferable for the polymeric material (e.g., polyurethane polycarbonate materials) that forms the implant cage (e.g., superior surface and/or inferior surface of the cage) to have a compressive modulus similar to that of the portion of the adjacent vertebrae with which they adjoin. For example, the compressive modulus of the polymeric material may be between 0.5 GPa and 1.5 GPa; and, in some cases, between 0.75 GPa and 1.25 GPa. The similar compressive modulus can lead to better load sharing between the implant cage and the adjacent vertebrae which can improve the biomechanical attributes of the implant cage.

In some embodiments, the implant cage includes a coating formed on surfaces of the cage. The coating, for example, may be a biomimetic and/or osteogenic (e.g., bone morphogenetic protein(s) (BMP) and related compounds) coating. In some cases, the coating can be used enhance bone growth on the cage. In some embodiments, the coating is formed on substantially all of the surfaces of the cage; though, in other embodiments, only a portion of the surfaces are coated; and, in some embodiments, the cage might not be coated at all. Suitable coating materials include calcium phosphate, BMP and related compounds, amongst others.

In some embodiments, a substance (e.g., a drug) may elute from the implant cage and/or a coating on the implant cage. For example, a substance incorporated into the implant cage and/or coating may be emitted into regions around the implant cage (e.g., within the windows). In some cases, the substance (e.g., BMP and related compounds) may be selected to enhance bone growth. The substance, for example, may be incorporated at different concentrations into different locations of the implant cage and/or coating.

Implant cage 10 may include fixation features 24 on one or more surfaces of the cage. These features can assist in immobilizing the cage within the vertebrae immediately after implantation and can further assist in resisting cage expulsion during use. In the illustrative embodiment, the fixation features are formed on the superior surface and the inferior surface. It should be understood that other embodiments may include fixation features on one, but not the other, of these surfaces.

Fixation features 24, as illustrated, are ridges formed by a series of a parallel peaks and valleys. In the illustrated embodiment, the ridges extend across a width (W) of the implant cage. Such a configuration of the fixation features has been shown to be particularly effective. It should be understood that other embodiments may include other types of fixation features.

In some embodiments, the fusion cage may include micro-texture features on the entire, or a portion of, the outer surface. The micro-texture features can be conducive to bone growth on the cage. For example, the feature sizes of the micro-texture may be between 1 micron and 10 microns; and, in some cases, may be between 1 micron and 6 microns.

In some embodiments, the fusion cage may include macro-texture features (e.g., protrusions) on the entire, or a portion of, the outer surface. The macro-texture features can form a series of troughs. In some embodiments, the macro-texture features may be formed on surfaces that define the windows. At least some of the troughs may be preferably oriented to extend from the superior surface to the inferior surface. The troughs, for example, may have a width of between 100 microns and 500 microns; a depth between 200 microns and 500 microns; and, may be separated from one another by a distance between 250 microns and 750 microns.

The implant cage can include one or more radiographic markers 26a, 26b which can be used to identify implant cage position for ensuring proper placement during implantation. For example, radiographic markers can be used for anterior-posterior orientation and lateral plane orientation. In general, the radiographic markers may be formed of any suitable material that is visible with x-rays systems and are compatible with the body. Suitable materials for the markers include certain metals (e.g., titanium, tantalum, gold, tungsten, platinum and mixtures thereof), other heavy elements (e.g., iodine) and polymeric materials loaded with a radio-opacifier (e.g., barium compounds including barium sulfate) or metal particles. In some embodiments, metal powder (e.g., micron-sized) may be mixed with a polymeric material (e.g., the polymeric material that is used to form the cage. The radiographic markers, for example, may be circular regions (e.g., having a diameter of less than 2 mm). In some cases, the radiographic markers are not relatively small discrete regions but, rather are diffused throughout larger regions of the fusion cage material. The radiographic markers may be present, for example, in amounts between 0.1 weight % and 5 weight % of the total weight of the cage; and, in some cases between 0.3 weight % and 1 weight % of the total weight of the cage.

In the illustrative embodiments, radiographic marker 26a is positioned on an outer surface of the anterior wall and radiographic marker 26b is positioned on an outer surface of the posterior wall. As shown, marker 26a is directly opposed from marker 26b.

It should be understood that other embodiments may not include any type of radiographic marker, or may include other types of radiographic markers and/or markers in different positions.

In general, the implant cage is anatomically shaped to fit between adjacent vertebrae. For example, the superior surface and the inferior surface can be curved and, for example, shaped to match anatomical curvatures on an adjacent vertebra. The curved surfaces can improve stress conditions within the cage once implanted. Furthermore, the curved surfaces can increase the cave surface area in contact with adjacent vertebra which can decrease the stress on the vertebra and reduce the potential for subsidence of the cage into the vertebra.

The anterior wall, posterior wall and sidewalls may include at least one tapered portion. As shown, tapered portion 28a of these walls extends downward from the superior surface at an outward angle; and, tapered portion 28b extends upward from the inferior surface at an outward angle. The taper angle may be between 0.1 degrees and 15 degrees; and, in some cases, between 0.5 degrees and 3 degrees. In the illustrated embodiment, the two tapered portions meet to define a waist portion which can be the maximum width of the cage. The outward angles may be the same as in some embodiments. The tapered portions may facilitate manufacturing of the implant cage using a molding process, as described further below. The waist portion may also lower stresses internal to the cage when placed under load once implanted. It should be understood that the presence of the tapered portions is optional.

In some embodiments, the fusion cage includes one or more features that are designed to cooperate with an insertion device. For example, as shown, the anterior wall includes holes 30a, 30b designed to cooperate with an insertion device. In the illustrative embodiment, the anterior wall also includes a protrusion 31 that is also designed to cooperate with an insertion device. It should be understood that the fusion cage may include no, or different types of, features that cooperate with the insertion device. Also, such features may be positioned in different locations on the cage.

In general, the inner wall is formed within the periphery defined by the anterior wall, the posterior wall, the first sidewall and the second sidewall. In the illustrative embodiment, inner wall 20 extends from substantially the center of the anterior wall to substantially the center of the posterior wall which may be preferred in some cases. However, in other embodiments, the inner wall may be positioned differently. The inner wall may provide mechanical support to the cage so that the cage retains its shape. Furthermore, the inner wall can increase the cage surface area in contact with adjacent vertebra which can decrease the stress on the vertebra and reduce the potential for subsidence of the cage into the vertebra.

In some embodiments, the cage includes a second inner wall contained within the periphery defined by the anterior wall, the posterior wall, the first sidewall and the second sidewall. In these embodiments, the cage may include one or more additional windows with the second inner wall defining a portion of at least one of the additional windows (e.g., a third window). The second inner wall may extend, for example, from the periphery to the first (e.g., center) inner wall.

As noted above, windows 22a, 22b extend from superior surface 12 at the top of the cage to inferior surface 14 at the bottom of the cage. One or more of the walls (e.g., the inner wall, the side wall, the anterior wall and the posterior wall) that define the windows extend straight vertically from the superior surface to the anterior surface. In some cases, all of the walls that define the windows extend straight vertically from the superior surface to the anterior surface. In these cases, the dimensions of the windows at the superior surface are generally the same as the dimensions of the window at the anterior surface.

In the illustrative embodiment, the first window and the second window have substantially similar shapes. For example, the first window and the second window can have substantially rectangular shapes as shown. It should be understood that, in some embodiments, the first window may have a different shape than the second window. Also, in some embodiments, one or more windows may have a non-rectangular shape.

It may be is desirable for the windows to provide sufficient area for bone to grow therethrough. For instance, the total volume of the windows can be between 15% and 80% of the total volume of the cage; and, in some cases, between 25% and 45% of the total volume of the cage.

In the illustrative embodiment, the cage includes only two of such windows; however other embodiments of the cage may include additional windows formed by additional walls as described above.

During use, the cage is implanted between adjacent vertebrae using suitable instrumentation. For example, the cage may be implanted between adjacent cervical vertebrae. In some embodiments, the cage may be attached (e.g., by screws) to a cervical plate which extends between adjacent vertebra. In these embodiments, the cage may be designed to include one or more features that enable such attachment. It should be understood that not all embodiments are attached to a cervical plate and that, in some embodiments, the fusion cage is positioned between adjacent vertebra without the use of any additional components.

In general, the implant cages are made using a suitable manufacturing process. For instance, when the cages are formed of polymeric material, a molding process may be used. The molding process may be an injection molding process or a reaction injection molding process. These processes generally involve injection of polymeric material precursors and/or polymeric material (e.g., polyurethane material) in a fluid state into a mold cavity which may be defined between two mold halves. In some embodiments (e.g., when the implant cage is formed of a polyurethane material) it may be preferable to use a molding process including certain aspects that are described in PCT Publication No. WO 02/11975 which is the publication of commonly-owned International Application Serial No. PCT/GB01/03441 which is incorporated by references in its entirety. After the polymeric material is solidified in the shape of the cage, the mold cavity is opened and the cage is removed from the mold. In some embodiments, the cage may include features which facilitate removal from the mold cavity such as the tapered wall portions, as described above.

Amongst other advantages, the implant cage can have excellent performance attributes when implanted, while being cost effective to manufacture (e.g., when formed of a polymeric material as described above). For example, the implant cage can be designed to withstand the forces experienced during implantation and to provide suitable stability. Furthermore, the implant cages can be durable and may retain their properties over long periods of time.

1. An intervertebral fusion implant cage designed to be implanted between adjacent vertebrae, the implant cage comprising:
a curved superior surface shaped to match anatomical curvatures of an adjacent vertebra;
a curved inferior surface shaped to match anatomical curvatures of an adjacent vertebra;
an anterior wall;
a posterior wall;
a first sidewall connecting the anterior wall to the posterior wall;
a second sidewall connecting the anterior wall to the posterior wall at a side opposite the first sidewall;
an inner wall extending from the anterior wall to the posterior wall;
a first window extending from the superior surface to the inferior surface and defined between the inner wall, the first and second sidewalls, the anterior wall and the posterior wall; and
a second window extending from the superior surface to the inferior surface and defined between the inner wall, the first and second sidewalls, the anterior wall and the posterior wall.

2. The implant cage of claim 1, wherein the implant cage is formed in an injection molding process.

3. The implant cage of claim 1, wherein the implant cage is formed in a reaction injection molding process.

4. The implant cage of claim 1, wherein the implant cage is formed of polymeric material.

5. The implant cage of claim 4, wherein the polymeric material comprises a polycarbonate urethane.

6. The implant cage of claim 4, wherein the polymeric material has a compressive modulus between 0.5 GPa and 1.5 GPa.

7. The implant cage of claim 1, wherein the implant cage includes a biomimetic and/or osteogenic coating.

8. The implant cage of claim 7, wherein the coating comprises calcium phosphate.

9. The implant cage of claim 7, wherein the coating comprises BMP or a related BMP compound.

10. The implant cage of claim 1, further comprising fixation features formed on the superior surface.

11. The implant cage of claim 1, further comprising fixation features formed on the inferior surface.

12. The implant cage of claim 11, wherein the fixation features are ridges extending in a direction across a width of the implant cage.

13. The implant cage of claim 1, further comprising at least one radiographic marker, wherein the radiographic marker comprises a mixture of radiopaque material and polymeric material.

14. The implant cage of claim 13, wherein a first radiographic marker is positioned on an outer surface of the anterior wall and a second radiographic marker is positioned on an outer surface of the posterior wall.

15. The implant cage of claim 1, wherein the anterior wall, the posterior wall and the sidewalls include at least one tapered portion.

16. The implant cage of claim 15, wherein a first tapered portion extends downward from the superior surface at an outer angle and a second tapered portion extends upward from the inferior surface at an outer angle.

17. The implant cage of claim 1, wherein the anterior wall includes one or more features designed to cooperate with an insertion device.

18. The implant cage of claim 17, wherein the one or more features are one or more holes formed in the anterior wall.

19. The implant cage of claim 1, wherein a height of the anterior wall is greater than a height of the posterior wall.

20. The implant cage of claim 1, wherein the first window and the second window have substantially similar shapes.

21. The implant cage of claim 1, wherein the first window and the second window have different shapes.

22. The implant cage of claim 1, further comprising a second inner wall contained within the periphery defined by the anterior wall, the posterior wall, the first sidewall and the second sidewall.

23. The implant cage of claim 1, wherein the second inner wall defines a portion of a third window.

24. The implant cage of claim 1, wherein the inner wall extends from substantially the center of the anterior wall to substantially the center of the posterior wall.

25. The implant cage of claim 1, wherein the total volume of the windows is between 25% and 45% of the total volume of the implant cage.

26. The implant cage of claim 1, wherein the walls that define the windows extend straight vertically from the superior surface to the anterior surface.

27. The implant cage of claim 1, wherein the implant cage is configured to attach to an anterior cervical plate positioned between the adjacent vertebrae.

28. The implant cage of claim 1, further comprising a protrusion on the anterior wall.
